(19) United States
(12) Patent Application Publication
(10) Pub. No.: US 2024/0197156 A1
(43) Pub. Date: Jun. 20, 2024

(54) TREATMENT INSTRUMENT FOR ENDOSCOPE

(71) Applicant: OLYMPUS MEDICAL SYSTEMS CORP., Tokyo (JP)

(72) Inventors: Hiromasa KATO, Tokyo (JP); Yusuke Shiota, Machida-shi (JP); Chika Miyajima, Hachioji-shi (JP); Kotaro Yamada, Tachikawa-shi (JP); Daiki Hagiwara, Kokubunji-shi (JP)

(21) Appl. No.: 18/540,161

(22) Filed: Dec. 14, 2023

Related U.S. Application Data
(60) Provisional application No. 63/387,612, filed on Dec. 15, 2022.

(51) Int. Cl. A61B 1/018; A61B 1/00; A61B 18/14

(52) U.S. Cl. CPC A61B 1/018; A61B 1/00094; A61B 18/1492; A61B 2017/00269

(57) ABSTRACT

A treatment instrument includes a tube with a conduit; a wire located in the tube so as to advance and retract in the tube, and through which a high frequency current is allowed to flow. The treatment instrument may also include an electrode connected to a distal end of the wire; and a distal end tip located at a distal end portion of the tube, the distal end tip configured to accommodate at least a part of the electrode. The distal end tip includes a passage that connects with an outer peripheral surface and an inner peripheral surface of the distal end tip. The passage connects with the conduit.

### CLAIM OF PRIORITY

Priority is claimed on U.S. Patent Provisional Application No. 63/387,612, filed Dec. 15, 2022, the content of which is incorporated herein by reference.

### TECHNICAL FIELD

The present disclosure relates to a treatment instrument for an endoscope.

### BACKGROUND

In endoscopic treatments such as endoscopic submucosal dissection (ESD), treatment instruments for an endoscope for incision and dissection such as high frequency knives and treatment instruments for an endoscope for hemostasis can be used.

### SUMMARY/OVERVIEW

When a treatment instrument for an endoscope is used to execute hemostasis, an operator feeds water to specify a hemostasis site. When hemostasis is executed after a hemostasis site is specified, there may be no liquid serving as an electrolyte around the hemostasis site. For this reason, before hemostasis is performed, an operator executes a treatment to suction a liquid around a tissue to be hemostatically treated. Japanese Unexamined Patent Application, First Publication No. 2009-233269 describes a treatment instrument for an endoscope that can execute water supply and suction.

However, a problem can arise in that when a liquid around a tissue to be hemostatically treated is suctioned, living tissue might be suctioned in addition to the liquid.

In view of the above circumstances, the present approach can be helpful, such as to provide a treatment instrument for an endoscope capable of suitably executing water supply and suction.

According to a treatment instrument for an endoscope related to a first aspect of the disclosure, the treatment instrument of an endoscope includes: a tube, the tube including a conduit, a wire located in the tube so as to advance and retract in the tube, and through which a high frequency current is allowed to flow, an electrode connected to a distal end of the wire; and, a distal end tip located at a distal end portion of the tube, the distal end tip configured to accommodate at least a part of the electrode, wherein the distal end tip includes a passage that connects with an outer peripheral surface and an inner peripheral surface of the distal end tip, and wherein the passage connects with the conduit.

### BRIEF DESCRIPTION OF DRAWINGS

FIG. 1 is an overall view of an endoscopic treatment system according to a first embodiment.

FIG. 2 is an overall view showing a treatment instrument of the endoscopic treatment system.

FIG. 3 is a perspective view of a distal end portion of the treatment instrument.

FIG. 4 is a perspective view of the distal end portion of the treatment instrument in which a distal end tip is transparently displayed.

FIG. 5 is a cross-sectional view of the distal end portion of the treatment instrument in which a rod is positioned at a first position.

FIG. 6 is a cross-sectional view of the distal end portion of the treatment instrument in which the rod is positioned at a second position.

FIG. 7 is a perspective view of a distal end portion of a treatment instrument in an endoscopic treatment system according to a second embodiment.

FIG. 8 is a perspective view of the distal end portion of the treatment instrument in which a distal end tip is transparently displayed.

FIG. 9 is a cross-sectional view of the distal end portion of the treatment instrument in which a rod is positioned at a first position.

FIG. 10 is a cross-sectional view of the distal end portion of the treatment instrument in which the rod is positioned at a second position.

FIG. 11 is a perspective view showing a modified example of a locking portion.

FIG. 12 is a perspective view of a distal end portion of a treatment instrument in an endoscopic treatment system according to a third embodiment.

FIG. 13 is a perspective view of the distal end portion of the treatment instrument in which a distal end tip is transparently displayed.

FIG. 14 is a cross-sectional view of the distal end portion of the treatment instrument in which a rod is positioned at a first position.

FIG. 15 is a cross-sectional view of the distal end portion of the treatment instrument in which the rod is positioned at a second position.

FIG. 16 is a cross-sectional view showing a modified example of a side hole of the distal end tip.

FIG. 17 is a cross-sectional view showing another modified example of the side hole.

FIG. 18 is a perspective view of a distal end portion of a treatment instrument in an endoscopic treatment system according to a fourth embodiment.

FIG. 19 is a perspective view of the distal end portion of the treatment instrument in which a distal end tip is transparently displayed.

FIG. 20 is a cross-sectional view of the distal end portion of the treatment instrument in which with a rod at a first position.

FIG. 21 is a cross-sectional view of the distal end portion of the treatment instrument in which the rod at a second position.

FIG. 22 is a perspective view of a distal end portion of a treatment instrument in an endoscopic treatment system according to a fifth embodiment.

FIG. 23 is a perspective view of the distal end portion of the treatment instrument in which a distal end tip is transparently displayed.

FIG. 24 is a cross-sectional view of the distal end portion of the treatment instrument.

FIG. 25 is a cross-sectional view along line X-X shown in FIG. 24.

FIG. 26 is a cross-sectional view of the distal end portion of the treatment instrument in which a rod protruding.

FIG. 27 is a cross-sectional view showing a modified example of a side hole of the distal end tip.

FIG. 28 is a cross-sectional view showing another modified example of the side hole.

FIG. 29 is a cross-sectional view showing a modified example of the distal end tip.

FIG. 30 is a cross-sectional view of a distal end portion of a treatment instrument in which a rod is positioned at a first position in an endoscopic treatment system according to a sixth embodiment.

FIG. 31 is a cross-sectional view of the distal end portion of the treatment instrument including a rod of another aspect.

FIG. 32 is a cross-sectional view of the distal end portion of the treatment instrument including a rod of another aspect.

FIG. 33 is a cross-sectional view of the distal end portion of the treatment instrument including a rod of another aspect.

FIG. 34 is a perspective view of a distal end portion of a treatment instrument in an endoscopic treatment system according to a seventh embodiment.

FIG. 35 is a perspective view of the distal end portion of the treatment instrument in which a distal end tip is transparently displayed.

FIG. 36 is a perspective view of the distal end portion of the treatment instrument in which the distal end tip is transparently displayed.

FIG. 37 is a cross-sectional view of the distal end portion of the treatment instrument in which a rod at a first position.

FIG. 38 is a cross-sectional view of the distal end portion of the treatment instrument in which the rod is positioned at a second position.

FIG. 39 is a front view of the distal end tip showing a modified example of a side hole of the distal end tip.

FIG. 40 is a front view of the distal end tip showing another modified example of a side hole of the distal end tip.

FIG. 41 is a front view of the distal end tip showing another modified example of a side hole of the distal end tip.

FIG. 42 is a perspective view of a distal end portion of a treatment instrument in an endoscopic treatment system according to an eighth embodiment.

FIG. 43 is a perspective view of the distal end portion of the treatment instrument in which a distal end tip or the like is transparently displayed.

FIG. 44 is a cross-sectional view of the distal end portion of the treatment instrument.

FIG. 45 is a cross-sectional view of the distal end portion of the treatment instrument with a rod protruding.

FIG. 46 is a front view of the distal end tip and a suction cover of the treatment instrument.

### DETAILED DESCRIPTION

### First Embodiment

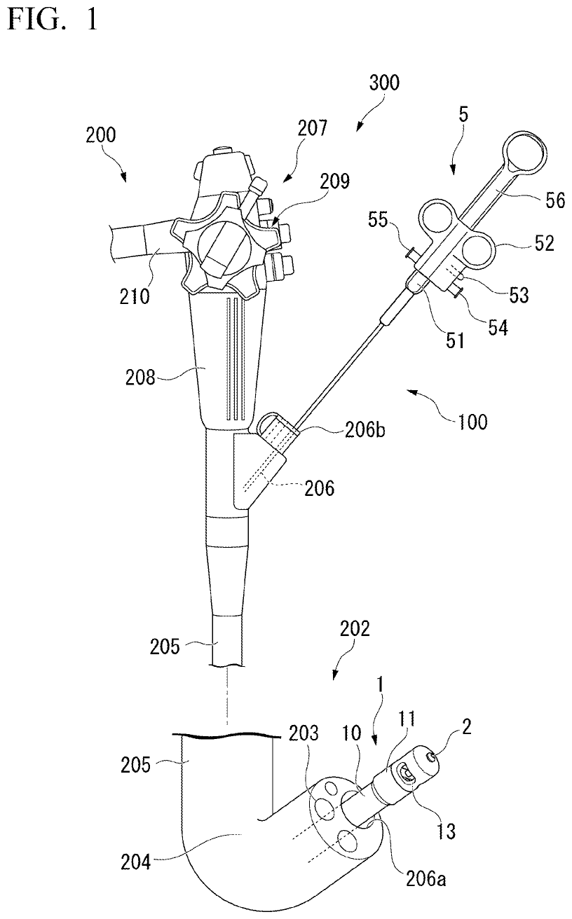

An endoscopic treatment system 300 according to a first embodiment of the present disclosure will be described with reference to FIGS. 1 to 6. FIG. 1 is an overall view of the endoscopic treatment system 300 according to the present embodiment.

### [Endoscopic Treatment System 300]

The endoscopic treatment system 300 includes an endoscope 200 and a treatment instrument 100, as shown in FIG. 1. The treatment instrument 100 is used by being inserted into the endoscope 200.

### [Endoscope 200]

The endoscope 200 is a known flexible endoscope and includes an insertion section 202 that is inserted into the body from its distal end, and an operation section 207 attached to the proximal end of the insertion section 202.

The insertion section 202 has an imaging section 203, a bending portion 204, and a flexible portion 205. The imaging section 203, the bending portion 204, and the flexible portion 205 are arranged in this order from the distal end of the insertion section 202. A channel 206 for inserting the treatment instrument 100 is provided inside the insertion section 202. A distal end opening portion 206a of the channel 206 is provided at the distal end of the insertion section 202.

The imaging section 203 includes an imaging element such as a CCD or a CMOS and is capable of imaging a site to be treated. The imaging section 203 is capable of imaging a rod 2 of the treatment instrument 100 in a state where the treatment instrument 100 protrudes from the distal end opening portion 206a of the channel 206.

The bending portion 204 bends in accordance with the operation of the operation section 207 by the operator. The flexible portion 205 is a tubular portion having flexibility.

The operation section 207 is connected to the flexible portion 205. The operation section 207 includes a grip 208, an input portion 209, a proximal end opening portion 206b of the channel 206, and a universal cord 210. The grip 208 is a part that is gripped by the operator. The input portion 209 receives an operation input for bending the bending portion 204. The universal cord 210 is for outputting an image captured by the imaging section 203 to the outside. The universal cord 210 is connected to a display device such as a liquid crystal display via an image processing device including a processor.

[Treatment instrument 100]

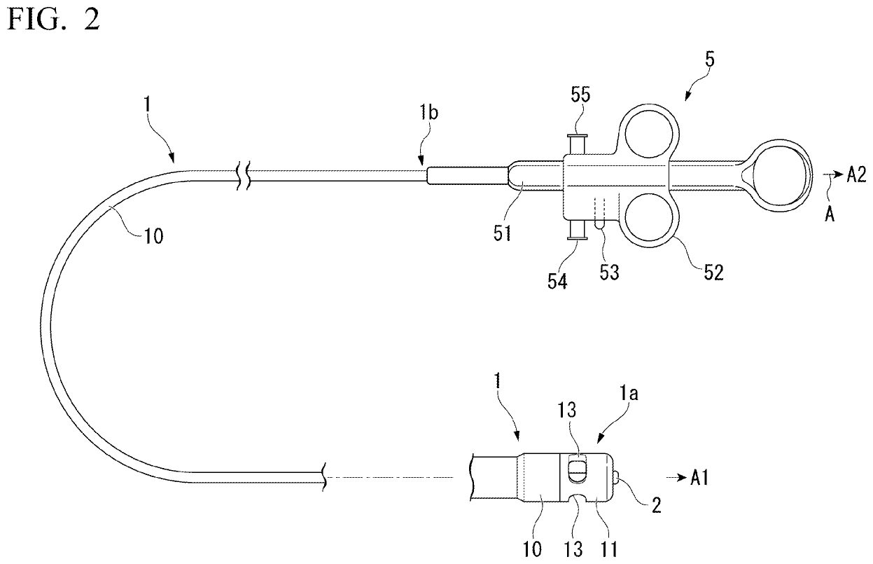

FIG. 2 is an overall view showing the treatment instrument 100.

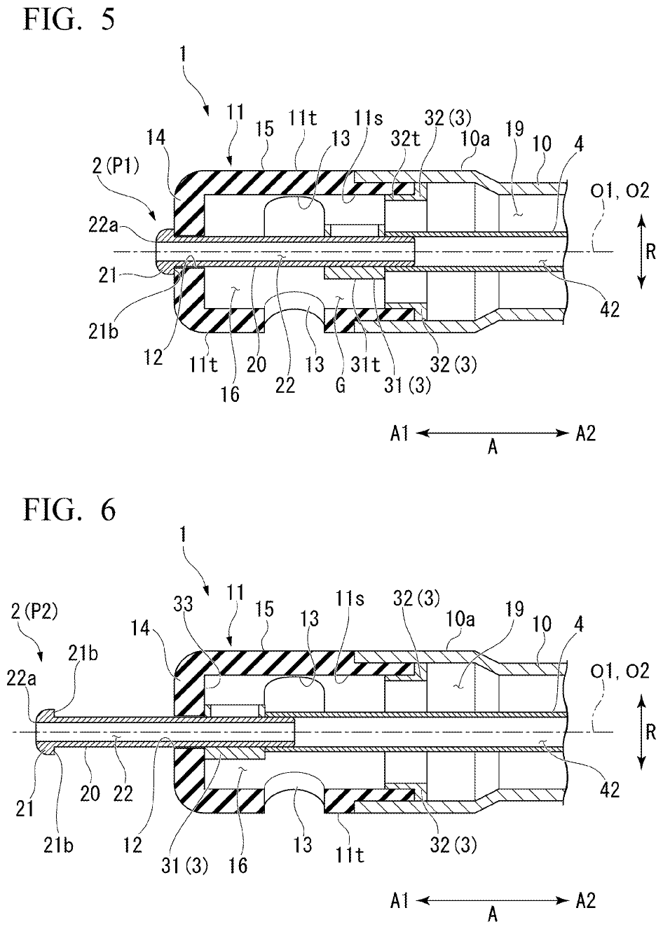

The treatment instrument (the treatment instrument for an endoscope) 100 is a multi-function type treatment instrument capable of executing local injection, incision, suction, coagulation, and hemostasis. The treatment instrument 100 includes a sheath 1, a rod 2, a stopper 3, an operation wire 4 (see FIG. 5), and an operation section 5. In the following description, in a longitudinal direction A of the treatment instrument 100, the side to be inserted into the patient's body is referred to as a “distal side A1,” and the side of the operation section 5 is referred to as a “proximal side A2.”

The sheath 1 is a long tubular member extending from a distal end 1a to a proximal end 1b. The sheath 1 has an outer diameter so as to be inserted into the channel 206 of the endoscope 200. The sheath 1 is configured to advance and retract through the channel 206. As shown in FIG. 1, in a state where the sheath 1 is inserted into the channel 206, the distal end 1a of the sheath 1 is capable of protruding and retracting from the distal end opening portion 206a of the channel 206.

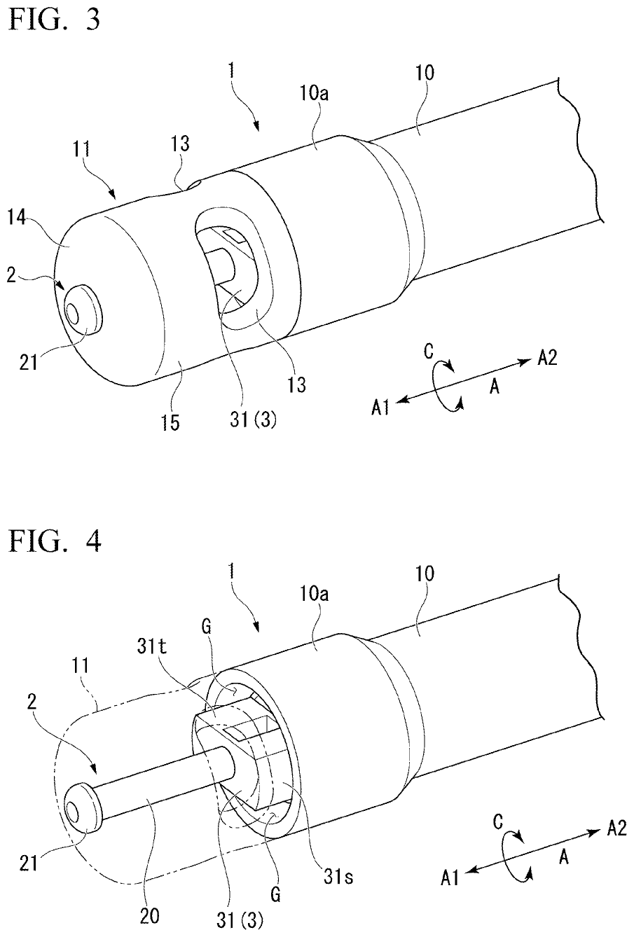

FIG. 3 is a perspective view of a distal end portion of the treatment instrument 100.

The sheath 1 has a tube 10 extending in the longitudinal direction A and a distal end tip 11 provided at a distal end of the tube 10. The sheath 1 may be formed by integrally molding the tube 10 and the distal end tip 11 with each other.

FIG. 4 is a perspective view of the distal end portion of the treatment instrument 100 in which the distal end tip 11 is transparently displayed.

The tube 10 is a long tubular member having flexibility and an insulation property. The tube 10 is, for example, formed of a resin. The distal end of the tube 10 is provided with an enlarged diameter portion 10a having an outer diameter larger than that of the proximal end side. The distal end tip 11 is fitted and attached to the inner side of the enlarged diameter portion 10a. The distal end tip 11 may be adhered to the enlarged diameter portion 10a with an adhesive or the like.

FIG. 5 is a cross-sectional view of the distal end portion of the treatment instrument 100 with the distal end of the rod 2 at a first position P1.

The distal end tip 11 is formed in a substantially cylindrical shape. The distal end tip 11 has a distal end portion 14 and a cylindrical portion 15. The distal end portion 14 is a substantially disk-shaped member extending in a radial direction R perpendicular to the longitudinal direction A at the distal end of the cylindrical portion 15. The cylindrical portion 15 is formed in a cylindrical shape with a central axis O1 of the sheath 1 in the longitudinal direction A as a central axis. A space surrounded by the distal end portion 14 and the cylindrical portion 15 is defined as an internal space 16 of the distal end tip 11. The distal end tip 11 is capable of accommodating at least a part of the rod 2 in the internal space 16. The internal space 16 of the distal end tip 11 communicates with an internal space (a conduit, a lumen) 19 of the tube 10.

A through hole 12 is formed in the distal end portion 14. The through hole 12 is a hole that is provided in the distal end portion 14 of the distal end tip 11 and penetrates the distal end portion 14 in the longitudinal direction A. The rod 2 is inserted through the through hole 12. The through hole 12 is provided at a position overlapping the central axis O1 of the sheath 1 in the longitudinal direction A. The position of the through hole 12 is not limited to this.

Three side holes (passages, absorption holes) 13 are formed in the cylindrical portion 15. The side holes (the passages, the absorption holes) 13 are holes that are provided in the cylindrical portion 15 of the distal end tip 11 and penetrate the cylindrical portion 15 in the radial direction R perpendicular to the longitudinal direction A. The side holes 13 are holes that penetrate a portion between an outer peripheral surface 11t and an inner peripheral surface 11s of the distal end tip 11. The three side holes 13 are provided evenly in a circumferential direction C. The number and aspect of the side holes 13 are not limited to this.

FIG. 6 is a cross-sectional view of the distal end portion of the treatment instrument 100 with the distal end of the rod 2 at a second position P2.

The rod (an electrode) 2 is a substantially round bar-shaped member made of a metal. The rod 2 is formed of a material such as stainless steel. The rod 2 has conductivity and is energized with a high frequency current. The rod 2 has a rod main body 20 and a flange 21.

The rod 2 is inserted through the through hole 12 of the distal end tip 11 of the sheath 1 in the longitudinal direction A and is capable of protruding from the through hole 12 toward the distal side A1. A central axis O2 of the rod 2 in the longitudinal direction A substantially coincides with the central axis O1 of the sheath 1 in the longitudinal direction A.

The rod main body 20 is a round bar-shaped member made of a metal. The operation wire 4 is attached to the proximal end of the rod main body 20. A high frequency current is supplied to the rod main body 20 from the operation wire 4 connected to the operation section 5. When a high frequency current is supplied from the operation wire 4 to the rod 2, the rod main body 20 and the flange 21 function as a monopolar electrode that output a high frequency current to a living tissue.

The flange (an enlarged diameter portion at the distal end) 21 is a disk-shaped conductive member provided at the distal end of the rod main body 20. In a front view seen in the longitudinal direction A, the outer periphery of the flange 21 is formed concentrically with the outer periphery of the rod main body 20. The length of the flange 21 in the radial direction R perpendicular to the longitudinal direction A is longer than the length of the rod main body 20 in the radial direction R. The flange 21 is not limited to a disk shape and may be a triangular shape or a hook shape.

A planar proximal end surface 21b is formed on the proximal side A2 of the flange 21. As shown in FIG. 5, when the rod 2 is retracted with respect to the sheath 1, the proximal end surface 21b of the rod 2 and the distal end portion 14 of the distal end tip 11 come into close contact with each other.

The rod main body 20 and the flange 21 have a first water supply conduit 22 extending in the longitudinal direction A. The first water supply conduit 22 communicates with a distal end opening 22a formed in the flange 21. The distal end opening 22a is an opening provided on the distal side A1 of the flange 21.

The stopper (a locking member) 3 is a member that restricts the advance and retraction movement range of the rod 2. The stopper 3 has a locking portion 31 and a locked portion 32.

As shown in FIG. 4, the locking portion 31 is formed in a substantially rectangular parallelepiped shape. The locking portion 31 is located on the proximal side A2 with respect to the distal end of the rod 2, and is provided on the outer peripheral surface of the rod main body 20. A sliding surface (a first outer peripheral surface) 31s is formed on the outer side of the locking portion 31 in the radial direction R along the inner peripheral surface 11s of the distal end tip 11. The locking portion 31 is capable of advancing and retracting in the internal space 16 of the distal end tip 11 in the longitudinal direction A while sliding the sliding surface 31s against the inner peripheral surface 11s of the distal end tip 11.

A gap G is formed between a part of the outer peripheral surface of the locking portion 31 and a part of the inner peripheral surface 11s of the distal end tip 11. Specifically, the gap G is formed between a second outer peripheral surface 31t recessed in the radial direction R with respect to the sliding surface (the first outer peripheral surface) 31s and a part of the inner peripheral surface 11s of the distal end tip 11. The gap G communicates with the side hole 13 and the internal space (the conduit, the lumen) 19 of the tube 10. The side hole 13, the gap G, and the internal space 19 form a suction flow passage. The second outer peripheral surface 31t may be a flat surface.

The locked portion (a first locked portion) 32 is an annular member provided pat a proximal end portion of the distal end tip 11, as shown in FIG. 5. The locked portion 32 is provided on the proximal side A2 with respect to the side hole 13. The locked portion 32 has a convex portion 32t that protrudes inward in the radial direction R with respect to the inner peripheral surface 11s of the distal end tip 11.

As shown in FIG. 5, when the distal end of the rod 2 is positioned at the first position P1, the locking portion 31 is engaged with the locked portion 32 (especially the convex portion 32t), and thus the retraction movement of the rod 2 is restricted. When the locking portion 31 is engaged with the locked portion 32, the proximal end surface 21b of the rod 2 and the distal end portion 14 of the distal end tip 11 come into close contact with each other.

As shown in FIG. 6, when the distal end of the rod 2 is positioned at the second position P2, the locking portion 31 is engaged with the proximal end surface of the distal end portion 14 of the distal end tip 11, and thus the advance movement of the rod 2 is restricted. The proximal end surface of the distal end portion 14 functions as a second locked portion 33 that restricts the advance movement of the rod 2. The second locked portion 33 is provided on the distal side A1 with respect to the side hole 13. When the locking portion 31 is engaged with the distal end portion 14 of the distal end tip 11, the locking portion 31 is disposed on the distal side A1 with respect to the side hole 13.

The operation wire 4 is a wire made of a metal which is inserted through the internal space (the conduit, the lumen) 19 of the tube 10. The operation wire 4 is formed of a material such as stainless steel. The distal end of the operation wire 4 is connected to the rod 2, and the proximal end of the operation wire 4 is connected to the operation section 5. The operation wire 4 may be in another aspect as long as it is a hollow shaft.

A second water supply conduit 42 is formed inside the operation wire 4. The second water supply conduit 42 is connected to and in fluid communication with the proximal end of the first water supply conduit 22.

The operation section 5 has an operation section main body 51, a slider 52, a power supply connector 53, a liquid supply port 54, and a suction connection port 55, as shown in FIGS. 1 and 2.

The distal end portion of the operation section main body 51 is connected to the proximal end 1b of the sheath 1. The operation section main body 51 has an internal space through which the operation wire 4 is configured to be inserted. The operation wire 4 passes through the internal space 19 of the tube 10 and the internal space of the operation section main body 51 and extends to the slider 52.

The slider 52 is attached movably in the longitudinal direction A with respect to the operation section main body 51. The proximal end portion of the operation wire 4 is attached to the slider 52. The operation wire 4 and the rod 2 are advanced and retracted in accordance with an operation in which the slider 52 advances and retracts by the slider 52 relative to the operation section main body 51.

The power supply connector 53 is fixed to the slider 52. The power supply connector 53 is capable of being connected to a high frequency power source apparatus (not shown) and is connected to the proximal end portion of the operation wire 4 via a conductive wire. The power supply connector 53 is configured to supply the high frequency current supplied from the high frequency power source apparatus to the rod 2 via the operation wire 4. The power supply connector 53 may be fixed to the operation section main body 51 instead of the slider 52.

The liquid supply port 54 is provided in the slider 52. The liquid supply port 54 is connected to a proximal end of the second water supply conduit 42 via a third water supply conduit formed in the slider 52. The liquid supplied from the liquid supply port 54 passes through the third water supply conduit, the second water supply conduit 42, and the first water supply conduit 22 and is discharged from the distal end opening 22a. The liquid supply port 54 may be provided in the operation section main body 51 instead of the slider 52.

The suction connection port 55 is provided in the slider 52. The suction connection port 55 is connected to the internal space (the conduit, the lumen) 19 of the tube 10. The suction connection port 55 can be connected to a suction pump (not shown). The suction is executed from the through hole 12 and the side hole 13 of the distal end tip 11 with the suction performed by the suction pump connected to the suction connection port 55.

[Method for using endoscopic treatment system 300]

Next, a procedure using the endoscopic treatment system 300 (a method of using the endoscopic treatment system 300) of the present embodiment will be described. Specifically, a local injection treatment, an incision and dissection treatment, and a hemostasis treatment for a lesion in endoscopic treatments such as endoscopic submucosal dissection (ESD) will be described.

As a preparation work, the operator specifies the lesion by a known method.

Specifically, the operator inserts the insertion section 202 of the endoscope 200 into the digestive tract (for example, the esophagus, the stomach, the duodenum, the large intestine) and specifies the lesion while observing the image obtained by the imaging section 203 of the endoscope.

### <Insertion Step>

The operator inserts the treatment instrument 100 into the channel 206 and protrudes the distal end 1a of the sheath 1 from the distal end opening portion 206a of the insertion section 202. The operator advances the slider 52 of the operation section 5 relative to the operation section main body 51 to protrude the rod 2.

### <Local Injection Step>

The operator punctures and penetrates a site where a liquid for local injection (a local injection liquid) is to be injected in the lesion with the rod 2. The operator supplies the liquid (the local injection liquid) from the liquid supply port 54 in a state where the distal end opening 22a at the distal end of the rod 2 is inserted into the submucosal layer.

### <Incision and Dissection Step>

Next, the operator performs an incision and dissection treatment. The operator advances the rod 2 and moves the flange 21 in a state where a high frequency current is applied to incise the mucosa of the lesion. In addition, the operator advances the rod 2 to dissect the submucosal layer of the incised lesion while lifting the mucosa of the incised lesion and exposing the submucosa layer in a state where a high frequency current is applied.

### <Hemostasis Step>

In a case where bleeding occurs during the incision and dissection treatment, the operator performs the hemostasis treatment. The operator supplies the liquid from the liquid supply port 54 to wash an area around a hemostasis point and specifies the hemostasis point. Next, the operator suctions the liquid, which is an electrolyte, from the surgical field in order to reliably perform the hemostasis treatment. The operator operates the suction pump connected to the suction connection port 55 to execute the suction from the through hole 12 and the side hole 13 of the distal end tip 11. In the distal end tip 11, the side hole 13 opens as a suction port in addition to the through hole 12, and thus it is prevented that the suction port is clogged by sucking the living tissue when the blood or the liquid for washing is suctioned in a state where the distal end of the distal end tip 11 is pressed against the living tissue.

Next, the operator cauterizes a bleeding point and stops bleeding by energizing the bleeding point with a high frequency current while pressing the rod main body 20 and the flange 21 against the bleeding point. At this time, since there is no liquid, which is an electrolyte, in the surgical field, the operator can reliably stop bleeding.

The operator continues the above-described operation (treatment) as necessary, finally excises the lesion, and ends the ESD procedure.

According to the treatment instrument 100 related to the present embodiment, it is possible to suitably execute water supply and suction. In the related art, the suction of the surgical field has been performed using the endoscope 200, but suction using the endoscope 200 is often accompanied by movement of the endoscope 200, and it is hard to continuously secure a visual field that captures the hemostasis point. According to the treatment instrument 100 related to the present embodiment, water supply, suction, and hemostasis is capable of being executed by the treatment instrument 100 while the endoscope 200 continuously secures a visual field that captures the hemostasis point.

In the above, the first embodiment of the present disclosure has been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Second Embodiment

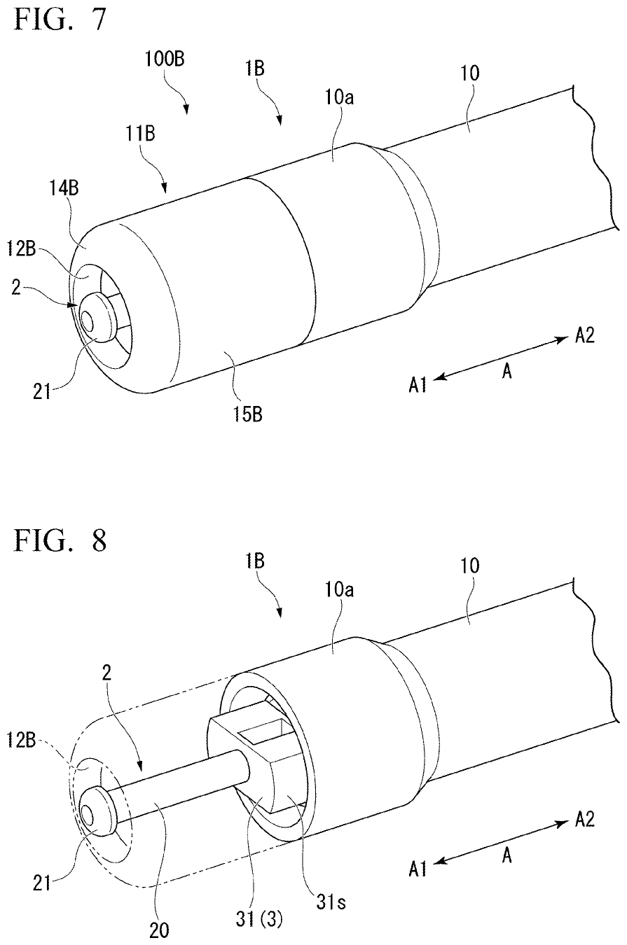

A treatment instrument 100B according to a second embodiment of the present disclosure will be described with reference to FIGS. 7 to 10. In the following description, the same constituent elements as those already described are designated by the same reference signs, and duplicate description will be omitted.

FIG. 7 is a perspective view of a distal end portion of the treatment instrument 100B.

The treatment instrument (the treatment instrument for an endoscope) 100B is a multi-function type treatment instrument capable of executing local injection, incision, suction, and coagulation and hemostasis. The treatment instrument 100B includes a sheath 1B, a rod 2, a stopper 3, an operation wire 4, and an operation section 5.

FIG. 8 is a perspective view of the distal end portion of the treatment instrument 100B in which a distal end tip 11B is transparently displayed.

The sheath 1B is a long tubular member extending from a distal end 1a to a proximal end 1b. The sheath 1B has an outer diameter so as to be inserted into the channel 206 of the endoscope 200. The sheath 1B is capable of advancing and retracting through the channel 206. The sheath 1B has a tube 10 extending in the longitudinal direction A and the distal end tip 11B provided at the distal end of the tube 10. The sheath 1B may be formed by integrally molding the tube 10 and the distal end tip 11B with each other.

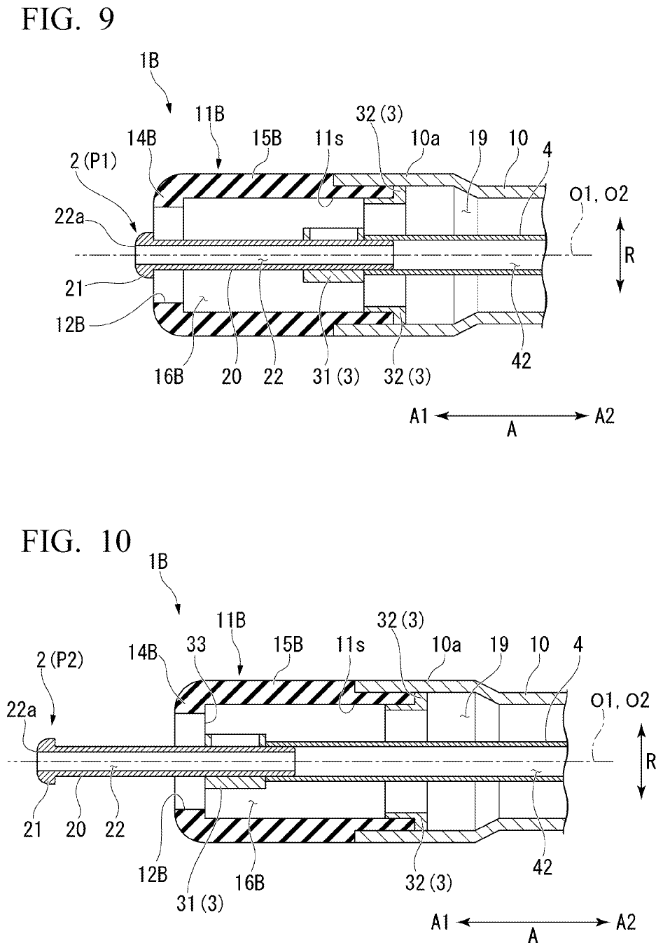

FIG. 9 is a cross-sectional view of the distal end portion of the treatment instrument 100B with the distal end of the rod 2 at a first position P1.

The distal end tip 11B is formed in a substantially cylindrical shape. The distal end tip 11B has a distal end portion 14B and a cylindrical portion 15B. The distal end portion 14B is a substantially annular member provided at the distal end of the cylindrical portion 15B. The cylindrical portion 15B is formed in a cylindrical shape with a central axis O1 of the sheath 1 in the longitudinal direction A as a central axis. A space surrounded by the distal end portion 14B and the cylindrical portion 15B is defined as an internal space 16B of the distal end tip 11B. The distal end tip 11B is capable of accommodating at least a part of the rod 2 in the internal space 16B. The internal space 16B of the distal end tip 11B communicates with an internal space (a conduit, a lumen) 19 of the tube 10.

As in the first embodiment, a locked portion (a first locked portion) 32 is an annular member provided at a proximal end portion of the distal end tip 11B, as shown in FIG. 9. The locked portion 32 has a convex portion 32t that protrudes inward in the radial direction R with respect to an inner peripheral surface 11s of the distal end tip 11B.

As shown in FIG. 9, when the distal end of the rod 2 is positioned at the first position P1, a locking portion 31 is engaged with the locked portion 32 (especially a convex portion 32t), and thus the retraction movement of the rod 2 is restricted. Even when the locking portion 31 is engaged with the locked portion 32, the distal end (a flange 21) of the rod 2 does not come into contact with the distal end tip 11B and is positioned to be spaced from the distal end tip 11B. In other words, even when the locking portion 31 is engaged with the locked portion 32, any part of the rod 2 may not contact with the distal end tip 11B and is spaced apart from the distal end tip 11B. In other words, even when the locking portion 31 is engaged with the locked portion 32, the rod 2 is spaced apart from an inner peripheral surface of the through hole 12B. Further, the distal end (the flange 21) of the rod 2 is supported by the locking portion 31 without contacting with the distal end tip 11B. The flange 21 may be completely accommodated in the internal space of the distal end tip 11B when the locking portion 31 is engaged with the locked portion 32. As another form, at least a part of the flange 21 may protrude from the distal end of the distal end tip 11B when the locking portion 31 is engaged with the locked portion 32.

A through hole 12B is formed in the distal end portion 14B. The through hole 12B is a hole that is provided in the distal end portion 14B of the distal end tip 11B and penetrates the distal end portion 14B in the longitudinal direction A. The rod 2 is inserted through the through hole 12B. The through hole 12B has a larger opening area than the through hole 12 of the first embodiment. The proximal end surface of the distal end portion 14B functions as a second locked portion 33 that restricts the advance movement of the rod 2, as in the first embodiment.

The cylindrical portion 15B does not have the side hole (the passage, the absorption hole) 13 unlike the cylindrical portion 15 of the first embodiment.

FIG. 10 is a cross-sectional view of the distal end portion of the treatment instrument 100B with the distal end of the rod 2 at a second position P2.

The rod 2 is inserted through the through hole 12B of the distal end tip 11B of the sheath 1B in the longitudinal direction A and configured to protrude from the through hole 12B toward the distal side A1. Even when the distal end of the rod 2 is disposed at any position in the advance and retraction movement range from the first position P1 to the second position P2, the flange 21 does not come into contact with the distal end tip 11B and is positioned to be spaced from the distal end tip 11B. The locking portion 31 is advanced and retract by sliding on the inner peripheral surface 11s of the distal end tip 11B. The locking portion 31 prevents the axis of the rod 2 from shaking in the longitudinal direction A. That is, the locking portion 31 supports the rod 2 in a cantilever manner. As a result, the central axis O2 of the rod 2 in the longitudinal direction A substantially coincides with the central axis O1 of the sheath 1B in the longitudinal direction A.

Further, even whether the distal end of the rod 2 is positioned at the first position P1 or the second position P2, the positions of the through hole 12B and the locking portion 31 are offset from each other in the longitudinal direction A. Specifically, when the distal end of the rod 2 is positioned at the first position P1, the locking portion 31 is located on the proximal side A2 with respect to the through hole 12B, and when the distal end of the rod 2 is positioned at the second position P2, the locking portion 31 is located on the distal side A1 with respect to the through hole 12B. For this reason, the locking portion 31 does not prevent the suction from the through hole 12B.

According to the treatment instrument 100B related to the present embodiment, it is possible to suitably execute water supply and suction. In the distal end tip 11B, the through hole 12B which has a larger opening area than the through hole 12 of the first embodiment opens as a suction port, and thus it is prevented that the suction port is clogged by sucking the living tissue when the blood or the liquid for washing is suctioned.

In the above, the second embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Modified Example

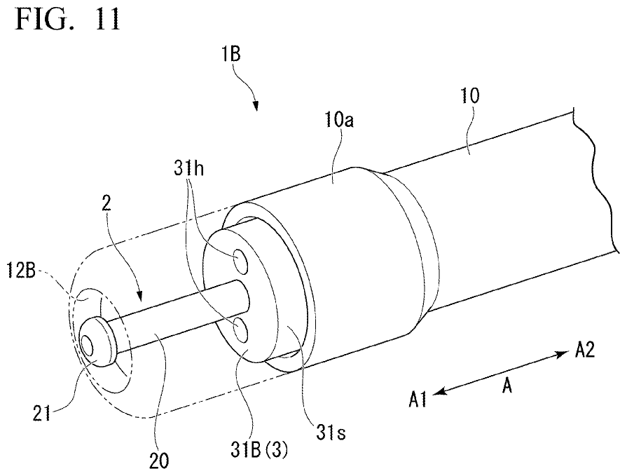

In the above embodiment, the gap between the locking portion 31 and the inner peripheral surface 11s of the distal end tip 11B is the suction flow passage. However, the suction flow passage is not limited to this. FIG. 11 is a perspective view showing a locking portion 31B that is a modified example of the locking portion 31. The locking portion 31B is formed in a circular column shape. A through hole 31h that penetrates the locking portion 31B in the longitudinal direction A is formed in the locking portion 31B. In this case, the through hole 31h is a main suction flow passage.

### Third Embodiment

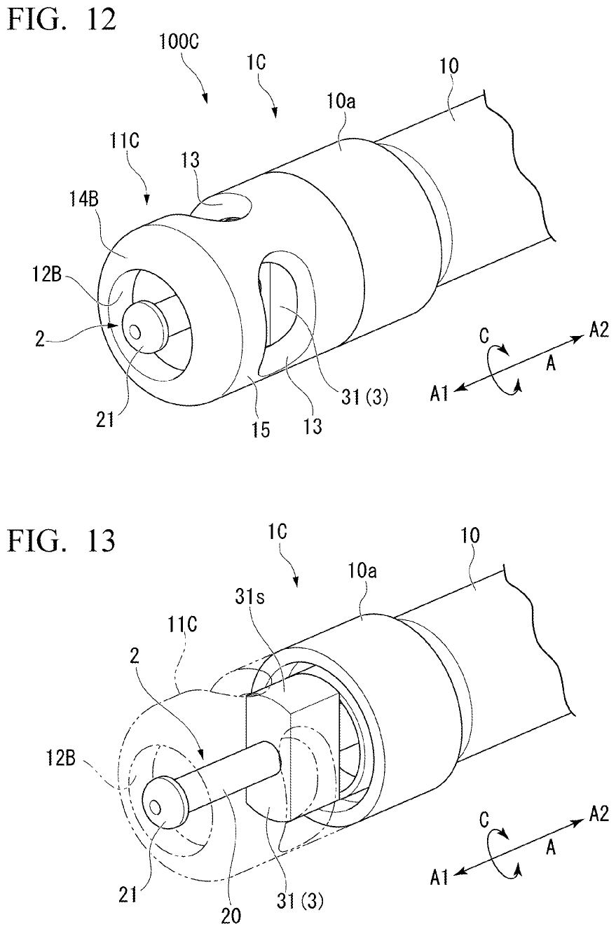

A treatment instrument 100C according to a third embodiment of the present disclosure will be described with reference to FIGS. 12 to 15. In the following description, the same constituent elements as those already described are designated by the same reference signs, and duplicate description will be omitted.

FIG. 12 is a perspective view of a distal end portion of the treatment instrument 100C.

The treatment instrument (the treatment instrument for an endoscope) 100C is a multi-function type treatment instrument capable of executing local injection, incision, suction, and coagulation and hemostasis. The treatment instrument 100C includes a sheath 1C, a rod 2, a stopper 3, an operation wire 4, and an operation section 5.

FIG. 13 is a perspective view of the distal end portion of the treatment instrument 100C in which a distal end tip 11C is transparently displayed. The sheath 1C is a long tubular member extending from a distal end 1a to a proximal end 1b. The sheath 1C has an outer diameter so as to be inserted into the channel 206 of the endoscope 200. The sheath 1C is capable of advancing and retracting through the channel 206. The sheath 1C has a tube 10 extending in the longitudinal direction A and the distal end tip 11C provided at the distal end of the tube 10. The sheath 1C may be formed by integrally molding the tube 10 and the distal end tip 11C with each other.

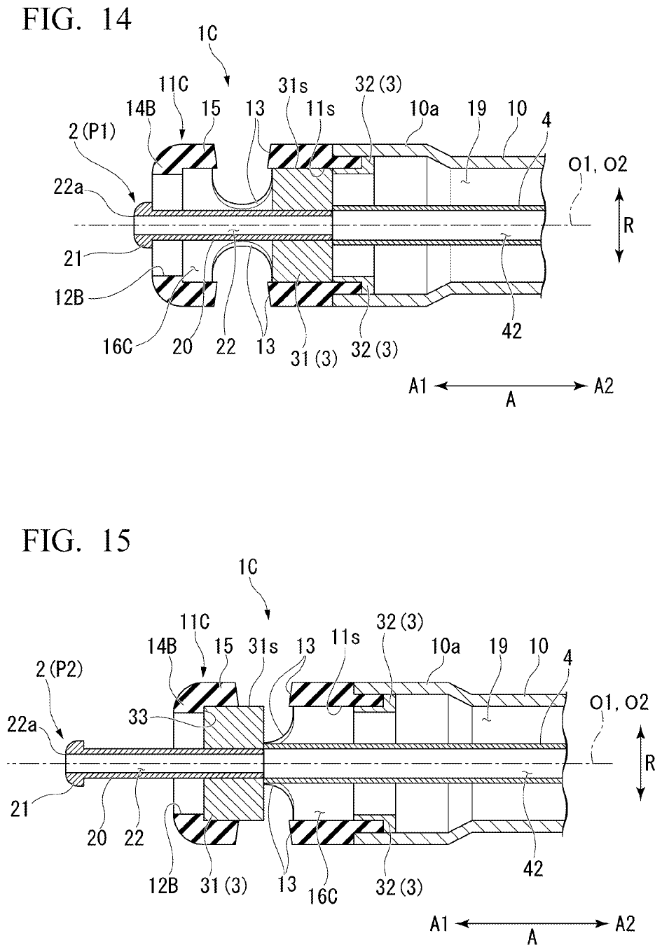

FIG. 14 is a cross-sectional view of the distal end portion of the treatment instrument 100C with the distal end of the rod 2 at a first position P1.

The distal end tip 11C is formed in a substantially cylindrical shape. The distal end tip 11C has a distal end portion 14B and a cylindrical portion 15. A space surrounded by the distal end portion 14B and the cylindrical portion 15 is defined as an internal space 16C of the distal end tip 11C. The distal end tip 11C can accommodate at least a part of the rod 2 in the internal space 16C. The internal space 16C of the distal end tip 11C communicates with an internal space (a conduit, a lumen) 19 of the tube 10.

As in the first embodiment, a locked portion (a first locked portion) 32 is an annular member provided at a proximal end portion of the distal end tip 11C, as shown in FIG. 14. The locked portion 32 has a convex portion 32t that protrudes inward in the radial direction R with respect to an inner peripheral surface 11s of the distal end tip 11C.

As shown in FIG. 14, when the distal end of the rod 2 is positioned at the first position P1, a locking portion 31 is engaged with the locked portion 32 (especially a convex portion 32t), and thus the retraction movement of the rod 2 is restricted. Even when the locking portion 31 is engaged with the locked portion 32, the distal end (a flange 21) of the rod 2 does not come into contact with the distal end tip 11C and is positioned to be spaced from the distal end tip 11C. Further, the distal end (the flange 21) of the rod 2 is supported by the locking portion 31 without contacting with the distal end tip 11C. The flange 21 may be completely accommodated in the internal space of the distal end tip 11C when the locking portion 31 is engaged with the locked portion 32. In addition, as another form, at least a part of the flange 21 may protrude from the distal end of the distal end tip 11C when the locking portion 31 is engaged with the locked portion 32.

FIG. 15 is a cross-sectional view of the distal end portion of the treatment instrument 100C with the distal end of the rod 2 at a second position P2.

The rod 2 is inserted through a through hole 12B of the distal end tip 11C of the sheath 1C in the longitudinal direction A. The rod 2 is capable of protruding from the through hole 12B toward the distal side A1. Even when the distal end of the rod 2 is disposed at any position in the advance and retraction movement range from the first position P1 to the second position P2, the flange 21 does not come into contact with the distal end tip 11C and is positioned to be spaced from the distal end tip 11C. The locking portion 31 is advanced and retract by sliding on the inner peripheral surface 11s of the distal end tip 11C. The locking portion 31 prevents the axis of the rod 2 from shaking in the longitudinal direction A. As a result, the central axis O2 of the rod 2 in the longitudinal direction A substantially coincides with the central axis O1 of the sheath 1C in the longitudinal direction A.

According to the treatment instrument 100C related to the present embodiment, it is possible to suitably execute water supply and suction. In the distal end tip 11C, the through hole 12B which has a larger opening area than the through hole 12 of the first embodiment opens as a suction port in addition to a side hole 13, and it is prevented that the suction port is clogged by sucking the living tissue when the blood or the liquid for washing is suctioned in a state where the distal end of the distal end tip 11C is pressed against the living tissue.

In the above, the third embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Modified Example

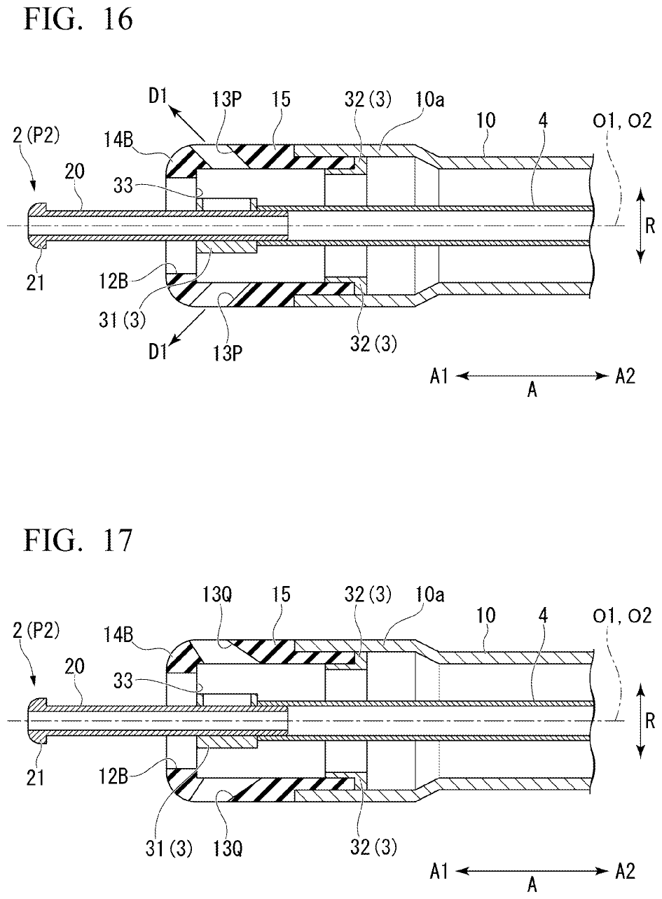

In the above embodiment, the side hole 13 is a hole formed in the radial direction R perpendicular to the longitudinal direction A. However, the shape of the side hole 13 is not limited to this. FIG. 16 is a cross-sectional view showing a side hole 13P that is a modified example of the side hole 13. The side hole 13P is a hole formed in a direction DI inclined with respect to the radial direction R. The direction DI is a direction in which the outer side of the radial direction R is inclined toward the distal side A1. FIG. 17 is a cross-sectional view showing a side hole 13Q that is a modified example of the side hole 13P. The side hole 13Q is formed in a tapered shape that widens from the outer side in the radial direction R toward the inner side in the radial direction R.

### Fourth Embodiment

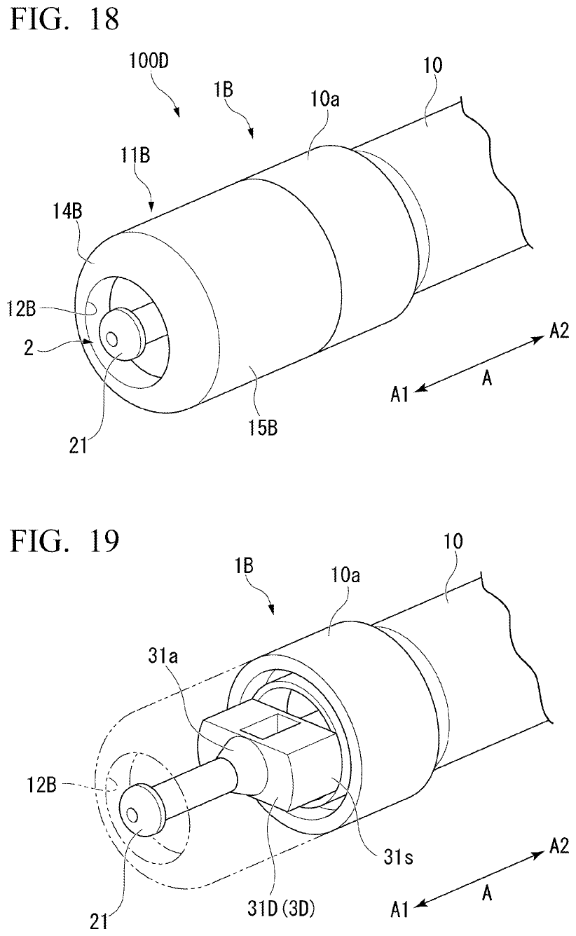

A treatment instrument 100D according to a fourth embodiment of the present disclosure will be described with reference to FIGS. 18 to 21. In the following description, the same constituent elements as those already described are designated by the same reference signs, and duplicate description will be omitted.

FIG. 18 is a perspective view of a distal end portion of the treatment instrument 100D.

The treatment instrument (the treatment instrument for an endoscope) 100D is a multi-function type treatment instrument capable of executing local injection, incision, suction, and coagulation and hemostasis. The treatment instrument 100D includes a sheath 1B, a rod 2, a stopper 3D, an operation wire 4, and an operation section 5.

FIG. 19 is a perspective view of the distal end portion of the treatment instrument 100D in which a distal end tip 11B is transparently displayed. The stopper 3D is a member that restricts the advance and retraction movement range of the rod 2. The stopper 3D has a locking portion 31D and a locked portion 32.

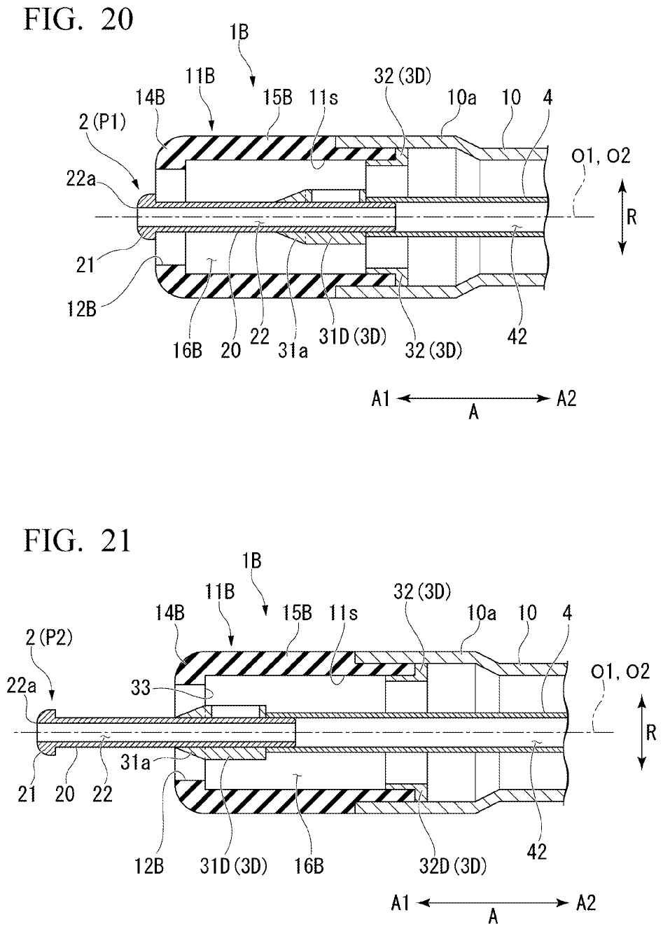

FIG. 20 is a cross-sectional view of the distal end portion of the treatment instrument 100D with the distal end of the rod 2 at a first position P1.

As shown in FIG. 19, the locking portion 31D is formed in a substantially rectangular parallelepiped shape and is provided on the outer peripheral surface of the rod main body 20 of the rod 2. A sliding surface 31s is formed on the outer side of the locking portion 31D in the radial direction R along the inner peripheral surface 11s of the distal end tip 11B. The locking portion 31D is capable of advancing and retracting in the internal space 16 of the distal end tip 11B in the longitudinal direction A while sliding the sliding surface 31s against the inner peripheral surface 11s of the distal end tip 11B.

FIG. 21 is a cross-sectional view of the distal end portion of the treatment instrument 100D with the distal end of the rod 2 at a second position P2.

The locking portion 31D has a pipe 31a for preventing the deflection of the rod 2 on the distal side A1. The pipe 31a is a tubular member provided on the distal side A1 of the locking portion 31D. The rod 2 is slidably provided in the pipe 31a. The outer shape of the pipe 31a may be conical, and the outer diameter of the pipe 31a on the distal side A1 is smaller than the outer diameter of the pipe 31a on the proximal side A2. The pipe 31a is capable of being inserted through a through hole 12B.

According to the treatment instrument 100D related to the present embodiment, it is possible to suitably execute water supply and suction. In the distal end tip 11B, the through hole 12B which has a larger opening area than the through hole 12 of the first embodiment opens as a suction port, and thus it is prevented that the suction port is clogged by sucking the living tissue even when the suction is continued. In addition, although the distal end of the rod 2 is spaced with respect to the distal end tip 11B, the deflection is suitably suppressed by the pipe 31a.

In the above, the fourth embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Fifth Embodiment

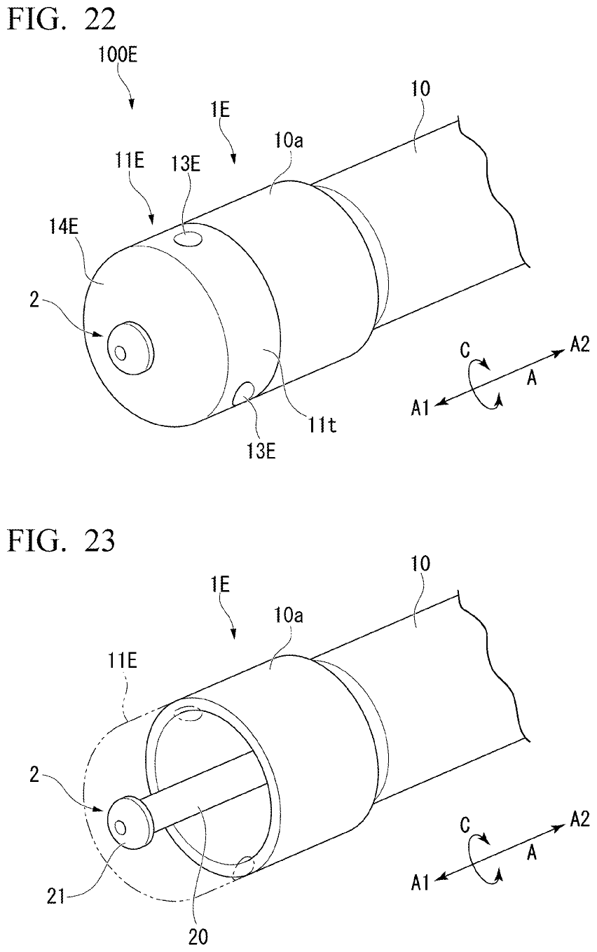

A treatment instrument 100E according to a fifth embodiment of the present disclosure will be described with reference to FIGS. 22 to 26. In the following description, the same constituent elements as those already described are designated by the same reference signs, and duplicate description will be omitted.

FIG. 22 is a perspective view of a distal end portion of the treatment instrument 100E.

The treatment instrument (the treatment instrument for an endoscope) 100E is a multi-function type treatment instrument capable of executing local injection, incision, suction, and coagulation and hemostasis. The treatment instrument 100E includes a sheath 1E, a rod 2, an operation wire 4, and an operation section 5.

FIG. 23 is a perspective view of the distal end portion of the treatment instrument 100E in which a distal end tip 11E is transparently displayed. The sheath 1E is a long tubular member extending from a distal end 1a to a proximal end 1b. The sheath 1E has an outer diameter so as to be inserted into the channel 206 of the endoscope 200. The sheath 1E is capable of advancing and retracting through the channel 206. The sheath 1E has a tube 10 extending in the longitudinal direction A and the distal end tip 11E provided at the distal end of the tube 10. The sheath 1E may be formed by integrally molding the tube 10 and the distal end tip 11E with each other.

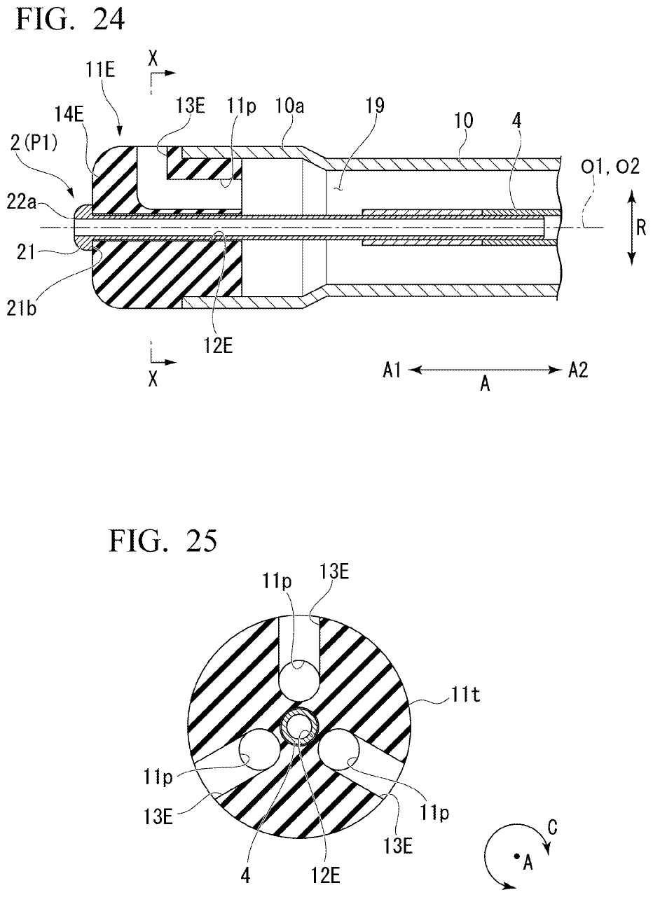

FIG. 24 is a cross-sectional view of the distal end portion of the treatment instrument 100E.

The distal end tip 11E is formed in a substantially circular column shape. The distal end tip 11E has a through hole 12E that penetrates the distal end tip 11E in the longitudinal direction A and three side holes 13E. The distal end tip 11E can accommodate at least a part of the rod 2 in the through hole 12E. The through hole 12E of the distal end tip 11E communicates with an internal space (a conduit, a lumen) 19 of the tube 10.

As shown in FIG. 24, when the rod 2 is moved backward with respect to the sheath 1E, a proximal end surface 21b of the rod 2 and a distal end portion 14E of the distal end tip 11E come into close contact with each other.

FIG. 25 is a cross-sectional view along line X-X shown in FIG. 24.

The side hole 13E opens in the outer peripheral surface 11t of the distal end tip 11E in the radial direction R. The side hole 13E communicates with the internal space (the conduit, the lumen) 19 of the tube 10 via a conduit 11p that is formed inside the distal end tip 11E and is bent at 90 degrees. The three side holes 13E are provided evenly in the circumferential direction C. The number and aspect of the side holes 13E are not limited to this.

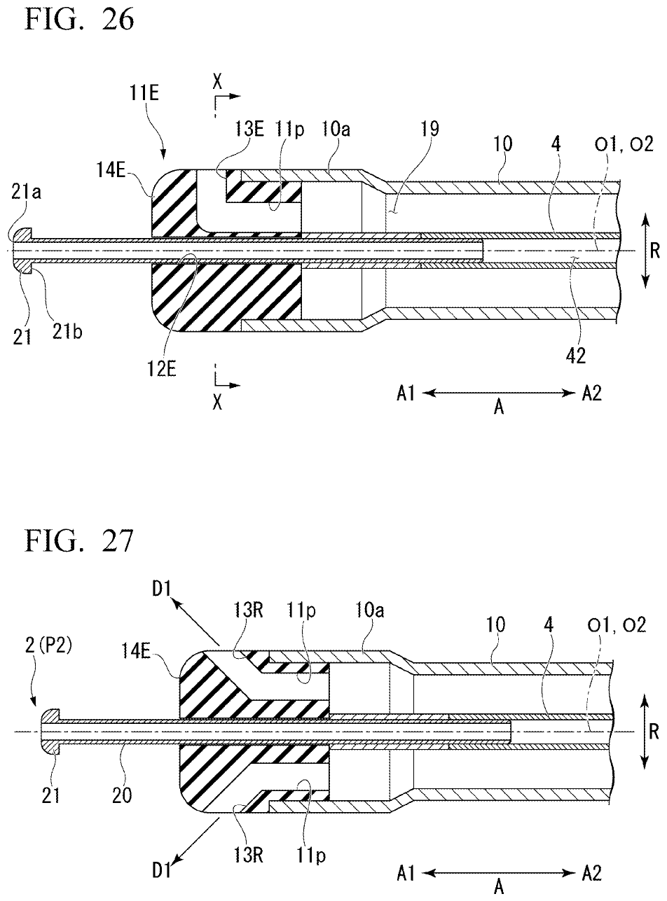

FIG. 26 is a cross-sectional view of the distal end portion of the treatment instrument 100E with the rod 2 protruding.

The rod 2 is inserted through the through hole 12E of the distal end tip 11E of the sheath 1E in the longitudinal direction A and is capable of advancing and retracting. A central axis O2 of the rod 2 in the longitudinal direction A substantially coincides with the central axis O1 of the sheath 1E in the longitudinal direction A.

According to the treatment instrument 100E related to the present embodiment, it is possible to suitably execute water supply and suction. In the distal end tip 11E, the side hole 13E opens as a suction port, and thus it is prevented that the suction port is clogged by sucking the living tissue when the blood or the liquid for washing is suctioned in a state where the distal end of the distal end tip 11E is pressed against the living tissue.

In the above, the fifth embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Modified Example

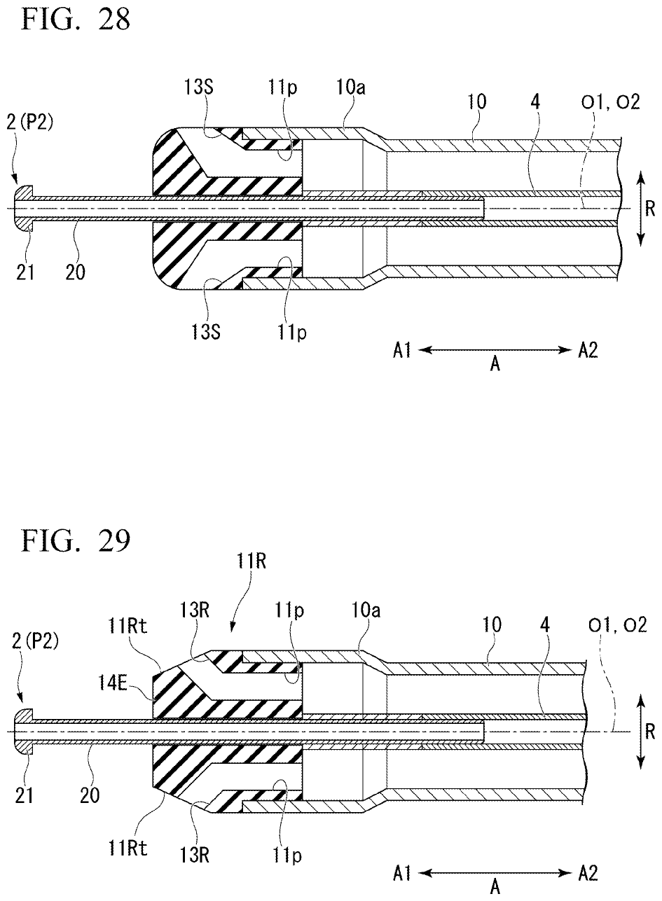

In the above embodiment, the side hole 13E is a hole formed in the radial direction R perpendicular to the longitudinal direction A. However, the shape of the side hole 13E is not limited to this. FIG. 27 is a cross-sectional view showing a side hole 13R that is a modified example of the side hole 13E. The side hole 13R is a hole formed in a direction DI inclined with respect to the radial direction R. The direction DI is a direction in which the outer side of the radial direction R is inclined toward the distal side A1. FIG. 28 is a cross-sectional view showing a side hole 13S that is a modified example of the side hole 13R. The side hole 13S is formed in a tapered shape that widens from the outer side in the radial direction R toward the inner side in the radial direction R. FIG. 29 is a cross-sectional view showing a distal end tip 11R that is a modified example of the distal end tip 11E. In the distal end tip 11R, an outer peripheral surface 11t provided with a side hole 13R is formed in a tapered shape that widens from the distal side A1 toward the proximal side A2 in the longitudinal direction A.

### Sixth Embodiment

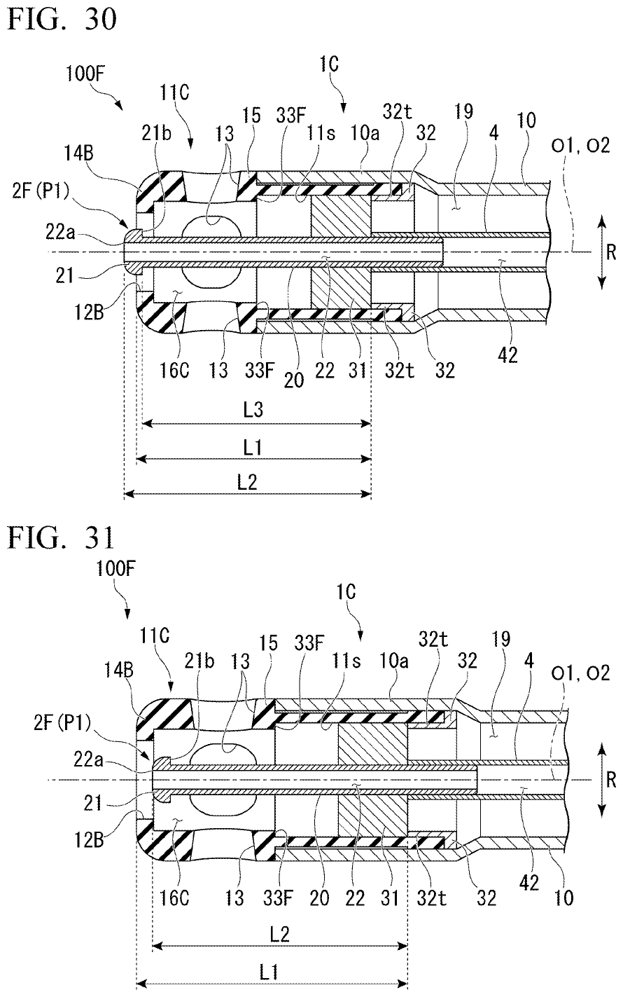

A treatment instrument 100F according to a sixth embodiment of the present disclosure will be described with reference to FIGS. 30 to 33. In the following description, the same constituent elements as those already described are designated by the same reference signs, and duplicate description will be omitted.

FIG. 30 is a cross-sectional view of a distal end portion of the treatment instrument 100F with the distal end of a rod 2F at a first position P1.

The treatment instrument (the treatment instrument for an endoscope) 100F is a multi-function type treatment instrument capable of executing local injection, incision, suction, and coagulation and hemostasis. The treatment instrument 100F includes a sheath 1C, the rod 2F, a stopper 3F, an operation wire 4, and an operation section 5.

The rod 2F is a substantially round bar-shaped member made of a metal. The rod 2F is formed of a material such as stainless steel. The rod 2F has conductivity and is energized with a high frequency current. The rod 2F has a rod main body 20F and a flange 21.

The stopper 3F is a member that restricts the advance and retraction movement range of the rod 2F. The stopper 3F has a locking portion 31, a locked portion 32, and a second locked portion 33F. The second locked portion 33F is provided on the distal side A1 with respect to the locked portion 32 and on the proximal side A2 with respect to a side hole 13. The second locked portion 33F locks with the locking portion 31 when the rod 2F is most advanced, that is, when the rod 2F is located at a second position P2.

In addition, when the distal end of the rod 2F is at the first position P1 or at the second position P2, the locking portion 31 is located on the proximal side A2 with respect to the side hole 13. For this reason, the locking portion 31 cannot prevent the suction from a through hole 12B and the side hole 13.

In the rod 2F shown in FIG. 30, a length L2 from the distal end surface of the flange 21 of the rod 2F to the distal end surface of a convex portion 32t of the locked portion 32 in the longitudinal direction A is longer than a length L1 from the distal end surface of a distal end portion 14B of a distal end tip 11C to the distal end surface of the convex portion 32t of the locked portion 32 in the longitudinal direction A. A length L3 from a proximal end surface 21b of the flange 21 of the rod 2F to the distal end surface of the convex portion 32t of the locked portion 32 in the longitudinal direction A is shorter than the length L1 (L3<L1<L2).

FIG. 31 is a cross-sectional view of the distal end portion of the treatment instrument 100E including the rod 2F of another aspect.

In the rod 2F shown in FIG. 31, the length L2 from the distal end surface of the flange 21 of the rod 2F to the distal end surface of the convex portion 32t of the locked portion 32 in the longitudinal direction A is shorter than the length L1 from the distal end surface of the distal end portion 14B of the distal end tip 11C to the distal end surface of the convex portion 32t of the locked portion 32 in the longitudinal direction A (L2<L1). For this reason, the flange 21 of the rod 2F at the first position P1 is accommodated in an internal space 16C. When the distal end of the rod 2F is at the first position P1 or at the second position P2, the locking portion 31 is located on the proximal side A2 with respect to the side hole 13. For this reason, the locking portion 31 cannot prevent the suction from the through hole 12B and the side hole 13.

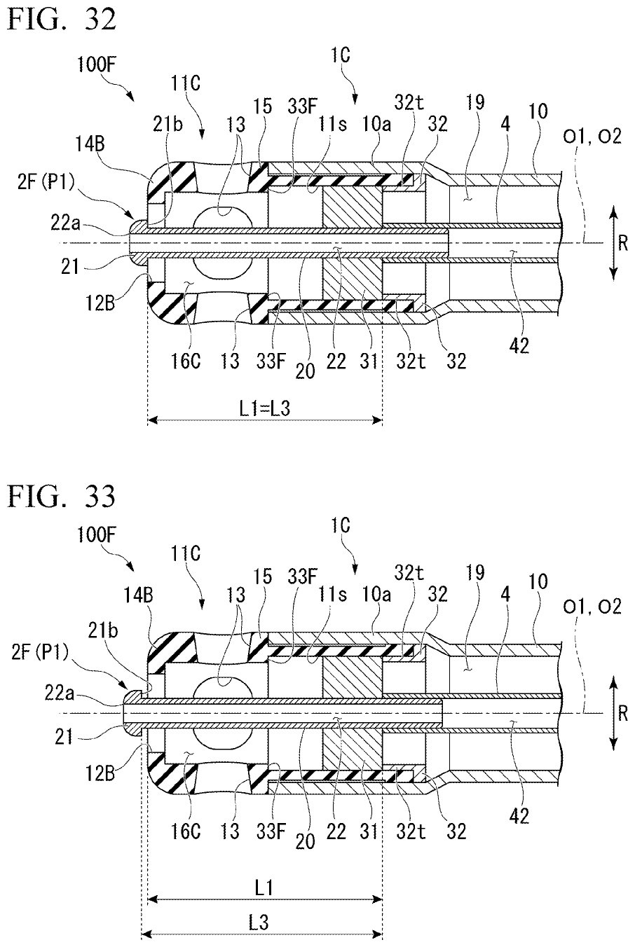

FIG. 32 is a cross-sectional view of the distal end portion of the treatment instrument 100E including the rod 2F of another aspect.

In the rod 2F shown in FIG. 32, the length L3 is substantially equal to the length L1 (L3=L1). The length L2 is longer than the length L1. When the distal end of the rod 2F is at the first position P1 or at the second position P2, the locking portion 31 is located on the proximal side A2 with respect to the side hole 13. For this reason, the locking portion 31 cannot prevent the suction from the through hole 12B and the side hole 13.

FIG. 33 is a cross-sectional view of the distal end portion of the treatment instrument 100E including the rod 2F of another aspect.

In the rod 2F shown in FIG. 33, the length L3 is longer than the length L1 (L1<L3). The length L2 is longer than the length L1. When the distal end of the rod 2F is at the first position P1 or at the second position P2, the locking portion 31 is located on the proximal side A2 with respect to the side hole 13. For this reason, the locking portion 31 cannot prevent the suction from the through hole 12B and the side hole 13.

In the above, the sixth embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Seventh Embodiment

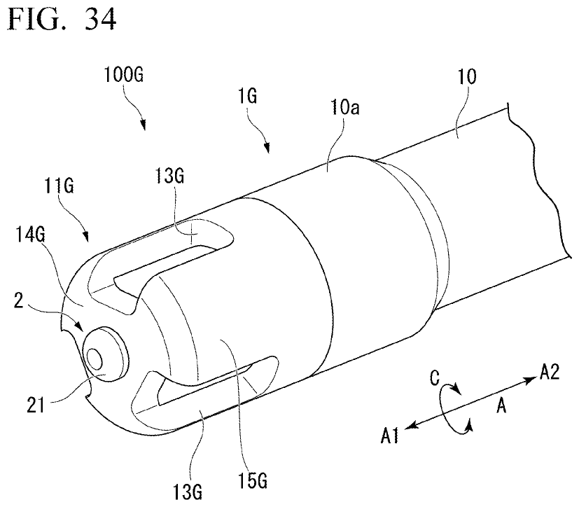

A treatment instrument 100G according to a seventh embodiment of the present disclosure will be described with reference to FIGS. 34 to 38. In the following description, the same constituent elements as those already described are designated by the same reference signs, and duplicate description will be omitted.

FIG. 34 is a perspective view of a distal end portion of the treatment instrument 100G.

The treatment instrument (the treatment instrument for an endoscope) 100G is a multi-function type treatment instrument capable of executing local injection, incision, suction, and coagulation and hemostasis. The treatment instrument 100G includes a sheath 1G, a rod 2, a stopper 3, an operation wire 4, and an operation section 5.

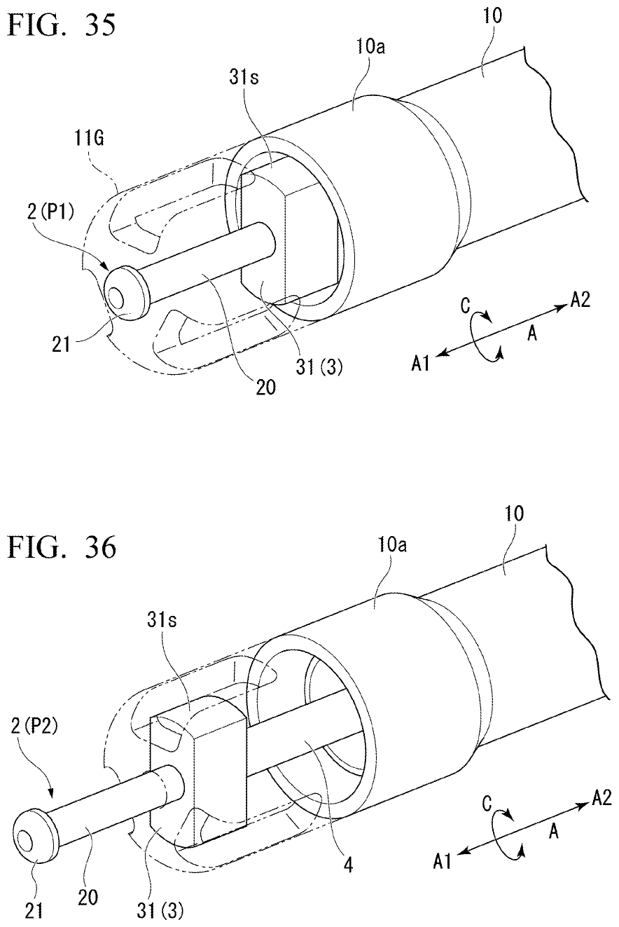

FIGS. 35 and 36 are perspective views of the distal end portion of the treatment instrument 100G in which a distal end tip 11G is transparently displayed. The sheath 1G is a long tubular member extending from a distal end 1a to a proximal end 1b. The sheath 1G has an outer diameter so as to be inserted into the channel 206 of the endoscope 200 and is capable of advancing and retracting through the channel 206. The sheath 1G has a tube 10 extending in the longitudinal direction A and the distal end tip 11G provided at the distal end of the tube 10. The sheath 1G may be formed by integrally molding the tube 10 and the distal end tip 11G with each other.

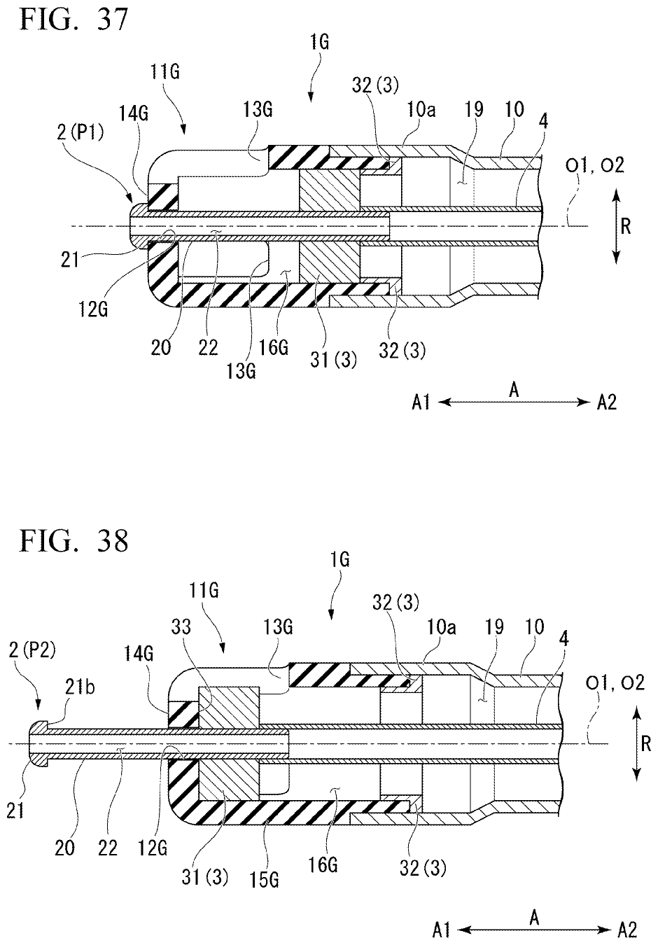

FIG. 37 is a cross-sectional view of the distal end portion of the treatment instrument 100G with the distal end of the rod 2 at a first position P1.

The distal end tip 11G is formed in a substantially cylindrical shape. The distal end tip 11G has a distal end portion 14G and a cylindrical portion 15G. The distal end portion 14G is a substantially disk-shaped member provided at the distal end of the cylindrical portion 15G. The cylindrical portion 15G is formed in a cylindrical shape with a central axis O1 of the sheath 1 in the longitudinal direction A as a central axis. A space surrounded by the distal end portion 14G and the cylindrical portion 15G is defined as an internal space 16G of the distal end tip 11G. The distal end tip 11G can accommodate at least a part of the rod 2 in the internal space 16G. The internal space 16G of the distal end tip 11G communicates with an internal space (a conduit, a lumen) 19 of the tube 10.

One through hole 12G is formed in the distal end portion 14G. The through hole 12G is a hole that is provided in the distal end portion 14G of the distal end tip 11G and penetrates the distal end portion 14G in the longitudinal direction A. The rod 2 is inserted through the through hole 12G. The through hole 12G is provided at a position overlapping the central axis O1 of the sheath 1 in the longitudinal direction A.

Three side holes (passages, absorption holes) 13G are formed in the cylindrical portion 15G. The side hole (the passage, the absorption hole) 13G is a hole that is provided in the cylindrical portion 15G of the distal end tip 11G and penetrates the cylindrical portion 15G in the radial direction R perpendicular to the longitudinal direction A. The side hole 13G is a hole that is formed to extend to the distal end portion 14G and penetrates the distal end portion 14G also in the longitudinal direction A. That is, the side hole 13G is a slit extending in the longitudinal direction A of the distal end tip 11G, and the distal end of the slit coincides with the distal end of the distal end tip 11G. The three side holes 13G are provided evenly in the circumferential direction C.

FIG. 38 is a cross-sectional view of the distal end portion of the treatment instrument 100G with the rod 2 at a second position P2.

The rod 2 is inserted through the through hole 12G of the distal end tip 11G of the sheath 1G in the longitudinal direction A and can protrude from the through hole 12G toward the distal side A1. A central axis O2 of the rod 2 in the longitudinal direction A substantially coincides with the central axis O1 of the sheath 1 in the longitudinal direction A. When the distal end of the rod 2 is positioned at the second position P2, the locking portion 31 is exposed from the side hole 13G.

According to the treatment instrument 100G related to the present embodiment, it is possible to suitably execute water supply and suction. In the distal end tip 11G, the side hole 13G which has a larger opening area than the side hole 13 of the first embodiment opens as a suction port, and thus it is prevented that the suction port is clogged by sucking the living tissue when the blood or the liquid for washing is suctioned in a state where the distal end of the distal end tip 11G is pressed against the living tissue.

In the above, the seventh embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

### Modified Example

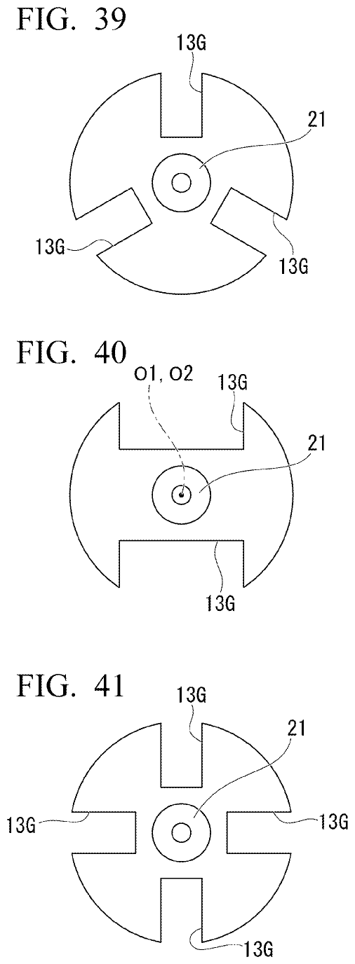

In the above embodiment, three side holes 13G are formed in the distal end tip 11G. However, the number and aspect of the side holes 13G formed in the distal end tip 11G are not limited to this. FIGS. 39, 40, and 41 are front views of the distal end tip 11G showing a modified example of the side hole 13G. As shown in FIG. 39, three side holes 13G may be evenly provided in the circumferential direction C. As shown in FIG. 40, two side holes 13G may be provided on both sides across the central axis O1. As shown in FIG. 41, four side holes 13G may be evenly provided in the circumferential direction C.

### Eighth Embodiment

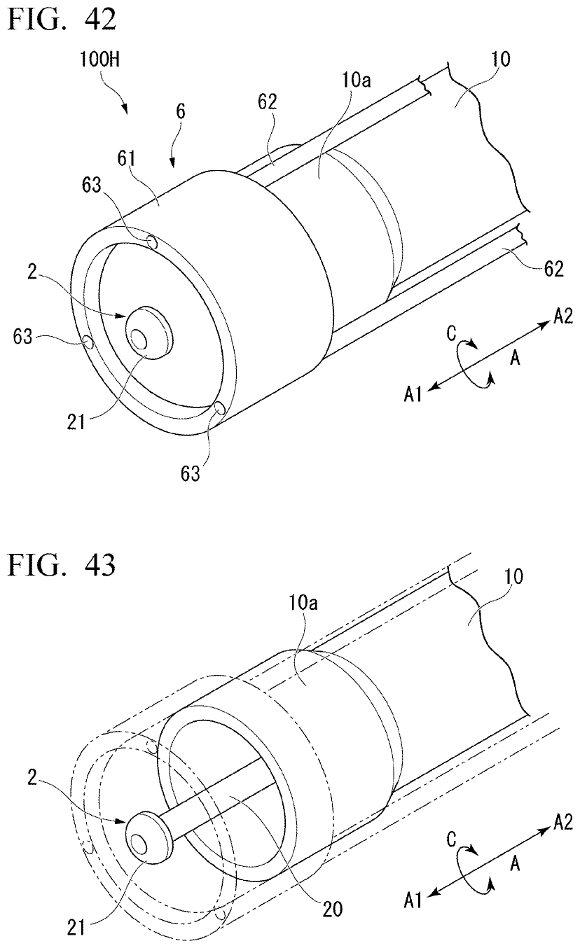

A treatment instrument 100H according to an eighth embodiment of the present disclosure will be described with reference to FIGS. 42 to 46. In the following description, the same constituent elements as those already described are designated by the same reference signs, and duplicate description will be omitted.

FIG. 42 is a perspective view of a distal end portion of the treatment instrument 100H.

The treatment instrument (the treatment instrument for an endoscope) 100H is a multi-function type treatment instrument capable of executing local injection, incision, suction, and coagulation and hemostasis. The treatment instrument 100H includes a sheath 1H, a rod 2, an operation wire 4, an operation section 5, and a suction cover 6.

FIG. 43 is a perspective view of the distal end portion of the treatment instrument 100H in which a distal end tip 11H or the like is transparently displayed. The sheath 1H is a long tubular member extending from a distal end 1a to a proximal end 1b. The sheath 1H has an outer diameter so as to be inserted into the channel 206 of the endoscope 200. The sheath 1H is capable of advancing and retracting through the channel 206. The sheath 1H has a tube 10 extending in the longitudinal direction A and the distal end tip 11H provided at the distal end of the tube 10. The sheath 1H may be formed by integrally molding the tube 10 and the distal end tip 11H with each other.

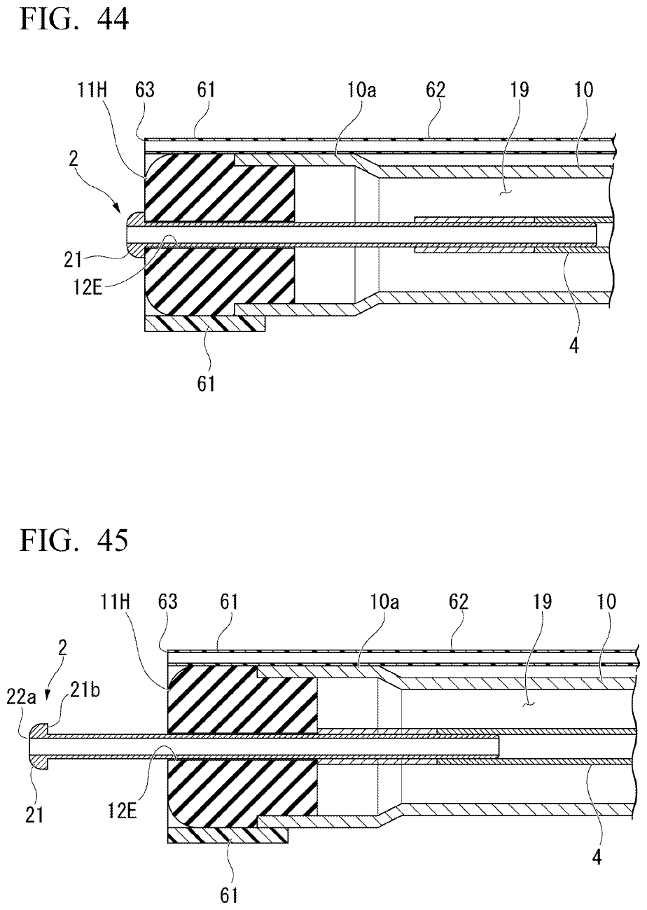

FIG. 44 is a cross-sectional view of the distal end portion of the treatment instrument 100H.

The distal end tip 11H is formed in a substantially circular column shape. The distal end tip 11H has a through hole 12E that penetrates the distal end tip 11H in the longitudinal direction A. The distal end tip 11H is capable of accommodating at least a part of the rod 2 in the through hole 12E. The through hole 12E of the distal end tip 11H communicates with an internal space (a conduit, a lumen) 19 of the tube 10.

The distal end tip 11H does not have the side hole (the passage, the absorption hole) 13E unlike the distal end tip 11E of the fifth embodiment.

FIG. 45 is a cross-sectional view of the distal end portion of the treatment instrument 100H with the rod 2 protruding.

The rod 2 is inserted through the through hole 12E of the distal end tip 11H of the sheath 1H in the longitudinal direction A and is capable of advancing and retracting. A central axis O2 of the rod 2 in the longitudinal direction A substantially coincides with the central axis O1 of the sheath 1H in the longitudinal direction A.

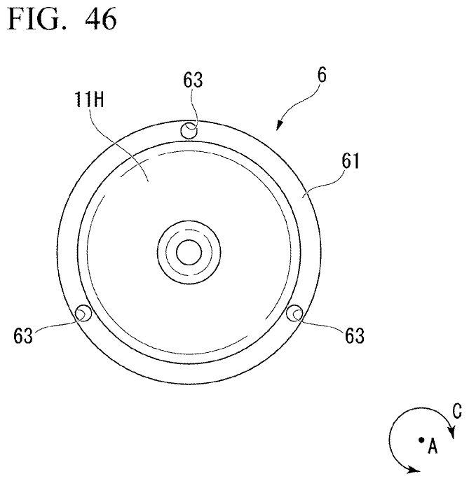

FIG. 46 is a front view of the distal end tip 11 and a suction cover 6.

The suction cover 6 has a cap 61 and three suction tubes 62. The cap 61 is formed in an annular shape and is attached to the outer peripheral surface of the distal end tip 11H. The three suction tubes 62 are tubes connected to the cap 61 and communicate with a distal end openings 63 provided on the distal side A1 of the cap 61. The distal end openings 63 are provided evenly in the circumferential direction C. The distal end openings 63 are disposed on the distal side A1 with respect to the distal end tip 11H. The suction tube 62 extends to the operation section 5 and configured to be connected to the suction pump (not shown).

According to the treatment instrument 100H related to the present embodiment, it is possible to suitably execute water supply and suction. Even in a case where the sheath 1H does not have a suction function, by attaching the suction cover 6, it is possible to suitably execute water supply and suction.

In the above, the eighth embodiment of the present disclosure have been described in detail with reference to the drawings, but the specific configuration is not limited to the embodiment, and a design change and the like within a range not departing from the scope of the present disclosure are also included. In addition, the constituent elements shown in the above-described embodiment and a modified example can be appropriately combined and configured.

While examples of the present disclosure have been described and illustrated above, it should be understood that these are exemplary of the disclosure and are not to be considered as limiting. Additions, omissions, substitutions, and other modifications can be made without departing from the spirit or scope of the present disclosure. Accordingly, the disclosure is not to be considered as being limited by the foregoing description, and is only limited by the scope of the appended claims.

What is claimed is:

1. A treatment instrument comprising:
a tube, the tube including a conduit;
a wire located in the tube so as to advance and retract in the tube, and through which a high frequency current is allowed to flow;
an electrode connected to a distal end of the wire; and
a distal end tip located at a distal end portion of the tube, the distal end tip configured to accommodate at least a part of the electrode,
wherein the distal end tip includes a passage that connects with an outer peripheral surface and an inner peripheral surface of the distal end tip, and wherein the passage connects with the conduit.

2. The treatment instrument according to claim 1, further comprising:
a locking portion located on an outer peripheral surface of the electrode and located proximally relative to a distal end of the electrode; and
a locked portion located at a proximal end portion of the distal end tip, wherein, when the electrode is disposed at a first position where the electrode is fully retracted, the locking portion and the locked portion are locked with each other, and the distal end of the electrode is spaced from the distal end tip.

3. The treatment instrument according to claim 2, wherein the locked portion is located proximally relative to the passage.

4. The treatment instrument according to claim 2, wherein the locked portion is a first locked portion, and
the treatment instrument further comprising:
a second locked portion configured to lock with the locking portion when the electrode is disposed at a second position where the electrode is fully advanced.

5. The treatment instrument according to claim 4, wherein the second locked portion is located distally relative to the passage.

6. The treatment instrument according to claim 4, wherein the second locked portion is located distally relative to the first locked portion and located proximally relative to the passage.

7. The treatment instrument according to claim 4, wherein the locking portion is slidable between the first locked portion and the second locked portion.

8. The treatment instrument according to claim 2, wherein a gap is formed between a part of an outer peripheral surface of the locking portion and a part of the inner peripheral surface of the distal end tip, and wherein the gap connects with the passage and the conduit.

9. The treatment instrument according to claim 2, wherein a gap is formed between the locking portion and a part of the inner peripheral surface of the distal end tip.

10. The treatment instrument according to claim 1, wherein the passage is a through hole extending between the inner peripheral surface and the outer peripheral surface of the distal end tip in a radial direction of the distal end tip.

11. A treatment instrument comprising:
a tube, the tube including a conduit;
a wire located in the tube so as to advance and retract in the tube and through which a high frequency current is allowed to flow;
an electrode connected to a distal end of the wire;
a distal end tip located at a distal end portion of the tube, the distal end tip configured to accommodate at least a part of the electrode;
a locking portion located on an outer peripheral surface of the electrode and located proximally relative to a distal end of the electrode; and
a locked portion located at a proximal end portion of the distal end tip, wherein, when the electrode is disposed at a first position where the electrode is fully retracted, the locking portion and the locked portion are locked with each other, and the distal end of the electrode is spaced from the distal end tip.

12. The treatment instrument according to claim 11, comprising a passage penetrating between an outer peripheral surface and an inner peripheral surface of the distal end tip.

13. The treatment instrument according to claim 12, wherein the locked portion is located proximally relative to the passage of the distal end tip.

14. The treatment instrument according to claim 12, wherein the locked portion is a first locked portion, and
the treatment instrument further comprising:
a second locked portion configured to lock with the locking portion when the electrode is disposed at a second position where the electrode is fully advanced.

15. The treatment instrument according to claim 14, wherein the second locked portion is located distally relative to the passage.

16. The treatment instrument according to claim 14, wherein the second locked portion is located distally relative to the first locked portion and located proximally relative to the passage.

17. The treatment instrument according to claim 14, wherein the locking portion is slidable between the first locked portion and the second locked portion.

18. The treatment instrument according to claim 12,
wherein a gap is formed between a part of an outer peripheral surface of the locking portion and a part of the inner peripheral surface of the distal end tip, and
wherein the gap connects with the passage and the conduit.

19. A treatment instrument comprising:
a tube, the tube including a conduit;
a wire located in the tube so as to of advance and retract in the tube and through which a high frequency current is allowed to flow;
an electrode connected to a distal end of the wire; and
a distal end tip with a passage connecting with an inner portion of the conduit and, the distal end tip configured to accommodate at least a part of the electrode,
wherein the electrode includes a locking portion provided on an outer peripheral surface of the electrode and located proximally relative to a distal end of the electrode, and
wherein the distal end tip includes a locked portion configured to lock with the locking portion and located proximally relative to the passage when the electrode is disposed at a first position where the electrode is fully retracted.

20. The treatment instrument according to claim 19, wherein, when the electrode is disposed at the first position, the distal end of the electrode is supported by the locking portion in a state that the distal end of the electrode is not in contact with the distal end tip.
